(19) United States
(12) Patent Application Publication
(10) Pub. No.: US 2007/0088331 A1
(43) Pub. Date: Apr. 19, 2007

(54) Method and apparatus for managing active agent usage, and active agent injecting device

(75) Inventors: Mizuo Nakayama, Shibuya-ku (JP); Takehiko Matsumura, Shibuya-ku (JP); Hidero Akiyama, Shibuya-ku (JP); Akihiko Matsumura, Shibuya-ku (JP)

Correspondence Address: SEED INTELLECTUAL PROPERTY LAW GROUP PLLC, 701 FIFTH AVE, SUITE 5400, SEATTLE, WA, 98104, US

(73) Assignee: Transcutaneous Technologies Inc., Shibuya-ku (JP)

(21) Appl. No.: 11/506,404

(22) Filed: Aug. 18, 2006

Related U.S. Application Data
(60) Provisional application No. 60/719,632, filed on Sep. 21, 2005.

(30) Foreign Application Priority Data
Aug. 18, 2005 (JP) 2005-237755

(51) Int. Cl. A61K 9/22

(52) U.S. Cl. 604/890.1; 604/500

(57) ABSTRACT

Whether or not a prescribed active agent is properly used may be confirmed. Usage of an active agent to be administered by an active agent injecting device (such as an iontophoresis device) driven by a drive signal may managed through a server based on actual drive information from the active agent injecting device.

### CROSS-REFERENCE TO RELATED APPLICATION

This application claims the benefit under 35 U.S.C. §119(e) of U.S. Provisional Patent Application No. 60/719,632, filed Sep. 21, 2005, and Japan Patent Application No. 2005-237755, filed Aug. 18, 2005, where these two applications are incorporated herein by reference in their entireties.

### BACKGROUND

1. Field

The present disclosure relates to a method and an apparatus for managing active agent usage, and an active agent injecting device. In particular, the present disclosure relates to a method and an apparatus for managing active agent usage, both of which may be used in managing usage of an active agent to be administered by an active agent injecting device, such as an iontophoresis device, that is driven by a drive signal. Usage of the active agent may be managed based on actual conditions of use. The present disclosure also related to an active agent injecting device that may be used along with the method and the apparatus.

2. Description

Conventionally, a subject may freely determine whether or not to take an active agent prescribed by a doctor. It has not been possible to confirm whether the subject has taken the prescribed active agent as indicated by the doctor. At present, at least half of the active agents prescribed by doctors and provided to subjects are thrown away, causing considerable waste and contributing to the problem of reducing medical expenses. This issue cannot be avoided as long as subjects themselves determine whether or not to use the prescribed active agent.

Iontophoresis has been proposed as a method of delivering an active agent into a subject through the skin or mucosa of the subject. An iontophoresis device may include an active electrode assembly having an active agent solution reservoir that holds an active agent solution, and a counter electrode assembly as a counter electrode to the active electrode assembly. An electric potential having the same polarity as that of an active agent ion in the active agent solution reservoir may be applied to the active electrode assembly so that the active agent solution is brought into contact with a biological interface, such as the skin or mucosa, of a subject in order to electrically drive active agent ions into the subject through the biological interface.

For example, WO 03/037425 A1 describes an iontophoresis device capable of stably administering an ionic active agent over a long period of time, while maintaining a high transport number. An active electrode assembly and a counter electrode assembly that comprise the iontophoresis device are both formed in a film state. Two or more ion exchange membranes having different ion selectivity are provided to the active electrode assembly in order to selectively pass or block ions. In addition, at least one ion exchange membrane is provided to the counter electrode assembly.

However, it has been conventionally considered to be difficult or impossible to manage active agent usage by a subject.

### BRIEF SUMMARY

In one aspect, the present disclosure is directed to managing usage of an active agent to be administered to a subject on the basis of the actual usage conditions.

In another aspect, the present disclosure is directed to an active agent injecting device suitable for managing usage of an active agent.

In one or more embodiments, usage conditions for an active agent to be administered by an active agent injecting device may be managed based on actual drive information from the device. It may thus become possible to confirm whether an active agent prescribed by a doctor has been properly used by a subject. The doctor may then be able to adequately understand the effectiveness of the amount and type of active agent used on the subject. In addition, disposal without administration of a portion of the active agent prescribed by the doctor can be eliminated, thus reducing medical waste and expense.

In one or more embodiments, the active agent injecting device may be adapted to validate a drive signal after authentication of the identity (ID) of a subject to whom an active agent is to be administered. Administration of the active agent can thus be limited to the subject for whom the agent has been prescribed.

In one or more embodiments, the ID of the subject may be provided to the subject in advance. As a result, erroneous input of the subject's ID can be prevented.

In one or more embodiments, the ID of the subject may be implanted in the body of the subject in advance. As a result, erroneous subject identifications may be reduced or prevented.

In one or more embodiments, the active agent injecting device may be an iontophoresis device. Usage of an active agent can thus be adequately managed.

In one or more embodiments, administration history may be managed through use of information stored in a server.

In one or more embodiments, the type, amount, and administration timing for an active agent administered to a subject can be managed for different subjects.

In one or more embodiments, a plurality of information about the administration of an active agent from an active agent injecting device may be collectively and easily input to a server.

In one or more embodiments, information about the administration of an active agent by an active agent injecting device may be input to a server in real time.

In one or more embodiments, an active agent injecting device may be suited to managing active agent usage because a drive signal is validated only after the ID of a subject, to whom an active agent is to be administered, has been authenticated.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is a block diagram that shows an embodiment of an apparatus for managing active agent usage.

FIG. 2 is a perspective view that shows a wrist band mounted with an integrated circuit (IC) chip.

FIG. 3 is a plan view that shows a first example of an iontophoresis device as an active agent injecting device.

FIG. 4 is an enlarged sectional view taken along the line IV-IV of FIG. 3.

FIG. 5 is an enlarged sectional view taken along the line V-V of FIG. 3.

FIG. 6 is a sectional view that shows a portion of a second example of an iontophoresis device.

FIG. 7 is a sectional view that shows a portion of a third example of an iontophoresis device.

FIG. 8 is a perspective view that shows an example of an apparatus that includes an active agent injecting device.

FIG. 9 is a perspective view that shows an example of a wrist band that includes an active agent injecting device.

FIG. 10 is a perspective view that shows an example of another apparatus that includes an active agent injecting device.

FIG. 11 is a flow chart that shows an operation example.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with iontophoresis devices, controllers, voltage or current sources and/or membranes have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment,” or “an embodiment,” or “another embodiment” means that a particular referent feature, structure, or characteristic described in connection with the embodiment is included in at least one embodiment. Thus, the appearances of the phrases “in one embodiment,” or “in an embodiment,” or “another embodiment” in various places throughout this specification are not necessarily all referring to the same embodiment. Further more, the particular features, structures, or characteristics may be combined in any suitable manner in one or more embodiments.

It should be noted that, as used in this specification and the appended claims, the singular forms “a,” “an,” and “the” include plural referents unless the content clearly dictates otherwise. Thus, for example, reference to a system for evaluating an iontophoretic active agent delivery including “a controller” includes a single controller, or two or more controllers. It should also be noted that the term “or” is generally employed in its sense including “and/or” unless the content clearly dictates otherwise.

As used herein the term “membrane” means a boundary, a layer, barrier, or material, which may, or may not be permeable. The term “membrane” may further refer to an interface. Unless specified otherwise, membranes may take the form a solid, liquid, or gel, and may or may not have a distinct lattice, non cross-linked structure, or cross-linked structure.

As used herein the term “ion selective membrane” means a membrane that is substantially selective to ions, passing certain ions while blocking passage of other ions. An ion selective membrane for example, may take the form of a charge selective membrane, or may take the form of a semi-permeable membrane.

As used herein the term “charge selective membrane” means a membrane that substantially passes and/or substantially blocks ions based primarily on the polarity or charge carried by the ion. Charge selective membranes are typically referred to as ion exchange membranes, and these terms are used interchangeably herein and in the claims. Charge selective or ion exchange membranes may take the form of a cation exchange membrane, an anion exchange membrane, and/or a bipolar membrane. A cation exchange membrane substantially permits the passage of cations and substantially blocks anions. Examples of commercially available cation exchange membranes include those available under the designators NEOSEPTA, CM-1, CM-2, CMX, CMS, and CMB from Tokuyama Co., Ltd. Conversely, an anion exchange membrane substantially permits the passage of anions and substantially blocks cations. Examples of commercially available anion exchange membranes include those available under the designators NEOSEPTA, AM-1, AM-3, AMX, AHA, ACH and ACS also from Tokuyama Co., Ltd.

As used herein, the term bipolar membrane means a membrane that is selective to two different charges or polarities. Unless specified otherwise, a bipolar membrane may take the form of a unitary membrane structure, a multiple membrane structure, or a laminate. The unitary membrane structure may include a first portion including cation ion exchange materials or groups and a second portion opposed to the first portion, including anion ion exchange materials or groups. The multiple membrane structure (e.g., two film structure) may include a cation exchange membrane laminated or otherwise coupled to an anion exchange membrane. The cation and anion exchange membranes initially start as distinct structures, and may or may not retain their distinctiveness in the structure of the resulting bipolar membrane.

As used herein, the term “semi-permeable membrane” means a membrane that is substantially selective based on a size or molecular weight of the ion. Thus, a semi-permeable membrane substantially passes ions of a first molecular weight or size, while substantially blocking passage of ions of a second molecular weight or size, greater than the first molecular weight or size. In some embodiments, a semi-permeable membrane may permit the passage of some molecules a first rate, and some other molecules a second rate different than the first. In yet further embodiments, the “semi-permeable membrane” may take the form of a selectively permeable membrane allowing only certain selective molecules to pass through it.

As used herein, the term “porous membrane” means a membrane that is not substantially selective with respect to ions at issue. For example, a porous membrane is one that is not substantially selective based on polarity, and not substantially selective based on the molecular weight or size of a subject element or compound.

As used herein and in the claims, the term “gel matrix” means a type of reservoir, which takes the form of a three dimensional network, a colloidal suspension of a liquid in a solid, a semi-solid, a cross-linked gel, a non cross-linked gel, a jelly-like state, and the like. In some embodiments, the gel matrix may result from a three dimensional network of entangled macromolecules (e.g., cylindrical micelles). In some embodiment a gel matrix may include hydrogels, organogels, and the like. Hydrogels refer to three-dimensional network of, for example, cross-linked hydrophilic polymers in the form of a gel and substantially composed of water. Hydrogels may have a net positive or negative charge, or may be neutral.

A used herein, the term “reservoir” means any form of mechanism to retain an element, compound, pharmaceutical composition, active agent, and the like, in a liquid state, solid state, gaseous state, mixed state and/or transitional state. For example, unless specified otherwise, a reservoir may include one or more cavities formed by a structure, and may include one or more ion exchange membranes, semi-permeable membranes, porous membranes and/or gels if such are capable of at least temporarily retaining an element or compound. Typically, a reservoir serves to retain a biologically active agent prior to the discharge of such agent by electromotive force and/or current into the biological interface. A reservoir may also retain an electrolyte solution.

A used herein, the term “active agent” refers to a compound, molecule, or treatment that elicits a biological response from any host, animal, vertebrate, or invertebrate, including for example fish, mammals, amphibians, reptiles, birds, and humans. Examples of active agents include therapeutic agents, pharmaceutical agents, pharmaceuticals (e.g., an active agent, a therapeutic compound, pharmaceutical salts, and the like) non-pharmaceuticals (e.g., cosmetic substance, and the like), a vaccine, an immunological agent, a local or general anesthetic or painkiller, an antigen or a protein or peptide such as insulin, a chemotherapy agent, an anti-tumor agent. In some embodiments, the term “active agent” further refers to the active agent, as well as its pharmacologically active salts, pharmaceutically acceptable salts, proactive agents, metabolites, analogs, and the like. In some further embodiment, the active agent includes at least one ionic, cationic, ionizable and/or neutral therapeutic active agent and/or pharmaceutical acceptable salts thereof. In yet other embodiments, the active agent may include one or more “cationic active agents” that are positively charged, and/or are capable of forming positive charges in aqueous media. For example, many biologically active agents have functional groups that are readily convertible to a positive ion or can dissociate into a positively charged ion and a counter ion in an aqueous medium. While other active agents may be polarized or polarizable, that is exhibiting a polarity at one portion relative to another portion. For instance, an active agent having an amino group can typically take the form an ammonium salt in solid state and dissociates into a free ammonium ion (NH₄⁺) in an aqueous medium of appropriate pH. The term “active agent” may also refer to neutral agents, molecules, or compounds capable of being delivered via electro-osmotic flow. The neutral agents are typically carried by the flow of, for example, a solvent during electrophoresis. Selection of the suitable active agents is therefore within the knowledge of one skilled in the art.

Non-limiting examples of such active agents include lidocaine, articaine, and others of the—caine class; morphine, hydromorphone, fentanyl, oxycodone, hydrocodone, buprenorphine, methadone, and similar opioid agonists; sumatriptan succinate, zolmitriptan, naratriptan HCl, rizatriptan benzoate, almotriptan malate, frovatriptan succinate and other 5-hydroxytryptamine1 receptor subtype agonists; resiquimod, imiquidmod, and similar TLR 7 and 8 agonists and antagonists; domperidone, granisetron hydrochloride, ondansetron and such anti-emetic active agents; zolpidem tartrate and similar sleep inducing agents; L-dopa and other anti-Parkinson's medications; aripiprazole, olanzapine, quetiapine, risperidone, clozapine and ziprasidone as well as other neuroleptica; diabetes active agents such as exenatide; as well as peptides and proteins for treatment of obesity and other maladies.

As used herein and in the claims, the term “subject” generally refers to any host, animal, vertebrate, or invertebrate, and includes fish, mammals, amphibians, reptiles, birds, and particularly humans.

The headings provided herein are for convenience only and do not interpret the scope or meaning of the embodiments.

Referring to FIG. 1, an apparatus for managing active agent usage may comprise: an active agent injecting device 120 that includes a switch 122 capable of being turned on by a command from a wired, or wireless, remote controller (hereinafter, “remote controller”) 124 after an identity (ID) of a subject to whom an active agent is to be administered, verified or otherwise confirmed or authenticated has been identified by, for example, a wireless integrated circuit (IC) tag or IC chip 112 implanted in a wrist band 110 mounted on a subject 100; and a server 130 that manages usage of an active agent to be administered by the active agent injecting device 120 based on actual drive information from the active agent injecting device 120.

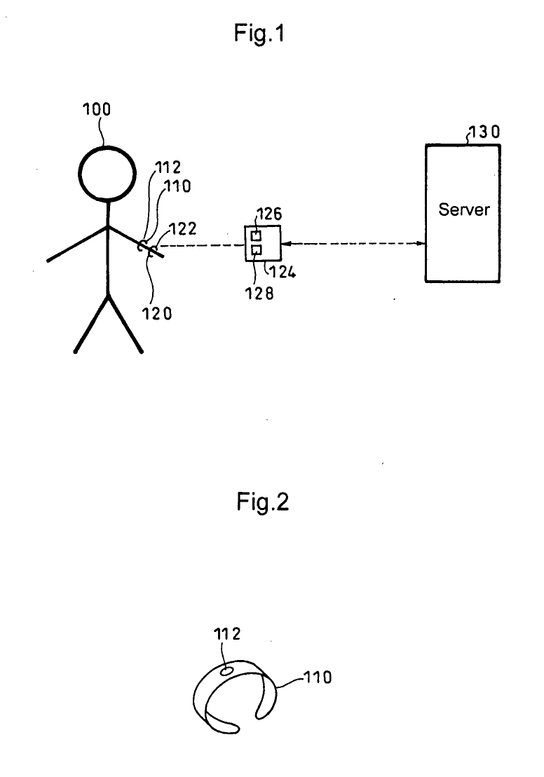

Referring to FIG. 2, the IC chip 112 may be provided to the subject 100 in the wrist band 110 or a bracelet, or by being implanted directly into the body of the subject 100. The erroneous description of information in the IC chip 112 can be prevented by configuring the chip to use an external electric power source, similar to Suica (registered trademark in Japan) or FeliCa (registered trademark in the US).

A subject ID information stored in the chip 112 may include one or more contraindicated active agents, disease names, and administration histories (e.g., active agent type (name), date(s) and time(s) of administration (e.g., year, month, day, hour, minute), and the amount of the active agent administered) for each subject as symbols for identifying the subject. The IC chip 112 may be extremely small, and thus easily implanted in the body of the subject. The IC chip 112 may also easily be adapted to be writable or rewritable by a reader/writer.

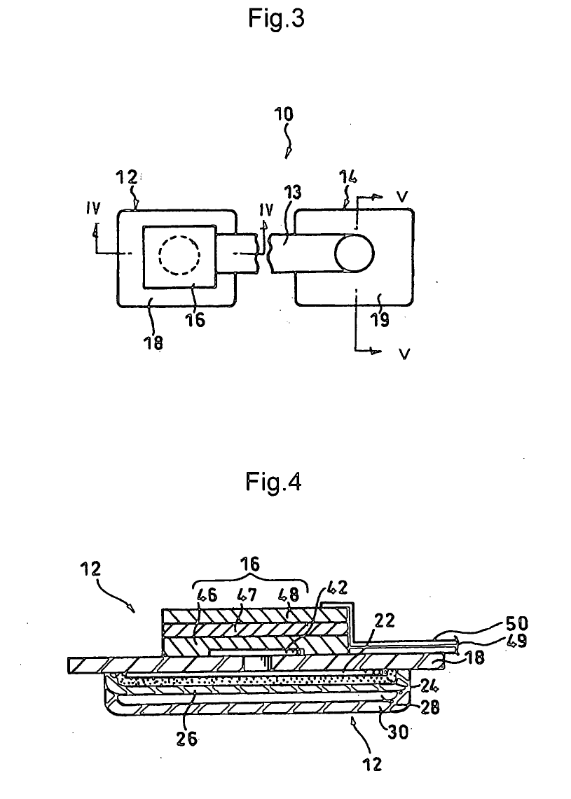

An iontophoresis device 10 shown in each of FIGS. 3 to 5 may be used as the active agent injecting device 120.

The iontophoresis device 10 comprises an active electrode assembly 12 and a counter electrode assembly 14 used for administering an ionic active agent, and a DC electric power source 16 connected to the electrode assemblies 12 and 14.

The active electrode assembly 12 comprises an active electrode 22, an electrolyte solution reservoir 24, a second ion exchange membrane 26, an active agent solution reservoir 28, and a first ion exchange membrane 30, in order from one surface of the base sheet 18 (the lower surface in FIG. 4).

The active electrode 22 may comprise a conductive paint blended with a non-metallic conductive filler, such as a carbon paste, and applied to the one surface of the base sheet 18. The active electrode 22 may also comprise a copper plate or a metallic thin film. However, metal may elute from the plate or the thin film and may transfer to a subject upon administration of an active agent. Therefore, the active electrode 22 is preferably nonmetallic.

The electrolyte solution reservoir 24 may comprise an electrolytic paint applied to the active electrode 22. The electrolytic paint is a paint containing an electrolyte. The electrolyte may be oxidized or reduced more readily than the electrolysis of water (oxidation on a positive electrode, reduction on a negative electrode). Examples of such electrolytes include: medical agents such as ascorbic acid (vitamin C) and sodium ascorbate; and organic acids such as lactic acid, oxalic acid, malic acid, succinic acid, and fumaric acid and/or salts thereof. The use of such an electrolyte may suppress the generation of oxygen and hydrogen gas. In addition, change in pH during operation of the iontophoresis device may be suppressed by blending a plurality of types of electrolytes to serve as a combination buffer electrolyte solution.

The electrolytic paint may be blended with a hydrophilic polymer such as polyvinyl alcohol, polyacrylic acid, polyacrylamide, or polyethylene glycol in order to improve the application property and film forming properties of the paint. The electrolytic paint may also be blended with a suitable amount of a solvent such as water, ethanol, or propanol to adjust viscosity. Further, the electrolytic paint may also comprise a suitable additional component such as a thickener, a thixotropic agent, a defoaming agent, a pigment, a flavor, and/or a coloring agent.

The second ion exchange membrane 26 may comprise an ion exchange resin into which an ion exchange group is introduced. Ions having a polarity opposite to that of active agent ions in the active agent solution reservoir 28, described below, may be used. If an active agent that dissociates into positive active agent ions is used in the active agent solution reservoir 28, the membrane is blended with an anion exchange resin. On the other hand, if an active agent that dissociates into negative active agent ions is used, the membrane is blended with a cation exchange resin.

The active agent solution reservoir 28 may comprise an active agent paint applied to the second ion exchange membrane 26. The paint may contain an active agent (or a precursor to the active agent) that dissociates into positive or negative active agent ions by dissolution in a solvent such as water. Examples of active agents that dissociate into positive active agent ions include lidocaine hydrochloride (an anesthetic) and morphine hydrochloride (an anesthetic). Examples of active agent that dissociate into negative active agent ions include ascorbic acid (a vitamin).

The first ion exchange membrane 30 may comprise a first ion exchange paint applied to the active agent solution reservoir 28. The first ion exchange paint may comprise an ion exchange resin into which ion exchange groups are introduced. The ion exchange groups have the same polarity as that of the active agent ion in the active agent solution reservoir 28. The paint may be blended with an anion or cation exchange resin when positive or negative active agent ions are used, respectively, in the active agent solution reservoir 28.

An ion exchange resin obtained by introducing a cation exchange group (an exchange group having a cation as a counter ion), such as a sulfonic group, a carboxylic group, or a phosphoric group, into a polymer having a three-dimensional network structure such as a hydrocarbon based resin (for example, a polystyrene resin or an acrylic resin) or a fluorine-based resin having a perfluorocarbon skeleton may be used as the cation exchange resin.

An ion exchange resin obtained by introducing an anion exchange group (an exchange group using an anion as a counter ion), such as a primary amino group, a secondary amino group, a tertiary amino group, a quaternary ammonium group, a pyridyl group, an imidazole group, a quaternary pyridinium group, or a quaternary imidazolium group, into a polymer having a three dimensional network structure similar to that of the cation exchange resin may be used as the anion exchange resin.

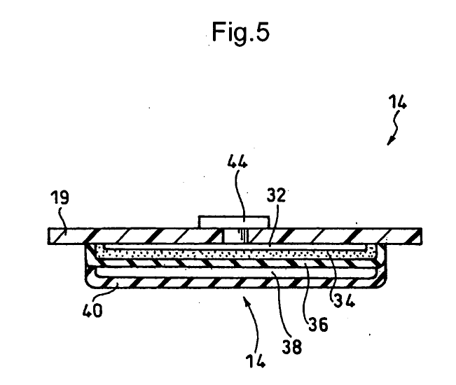

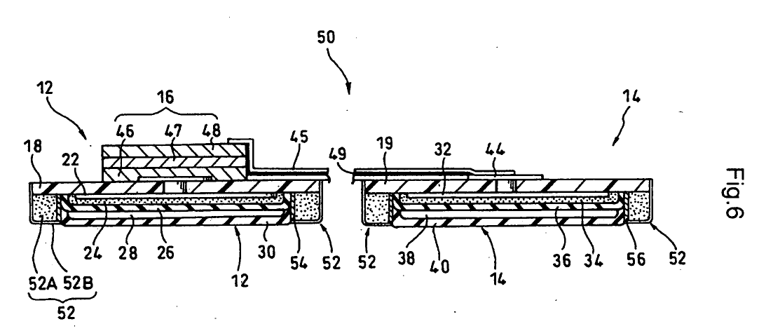

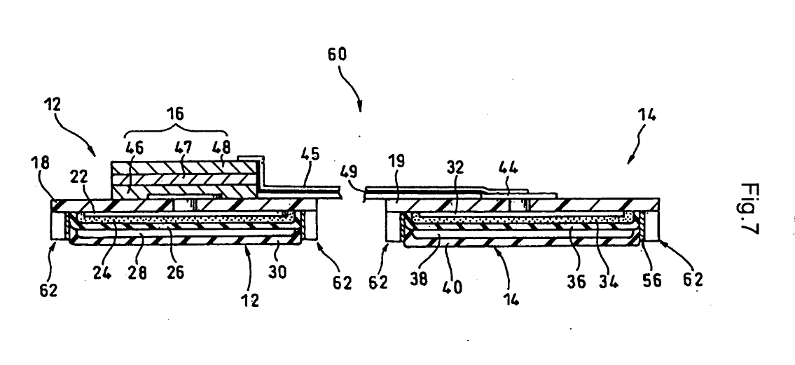

FIG. 5 is an enlarged sectional view that shows that the counter electrode assembly 14 may comprise a counter electrode 32, a second electrolyte solution reservoir 34, a third ion exchange membrane 36, a third electrolyte solution reservoir 38, and a fourth ion exchange membrane 40 laminated in this order on one surface of a counter base sheet 19, which is similar to the base sheet 18.

The counter electrode 32 may comprise a configuration similar to that of the active electrode 22 in the cell type active electrode assembly 12. In addition, the second electrolyte solution reservoir 34 may comprise the same electrolyte paint, which is applied to the counter electrode 32, as that of the electrolyte solution reservoir 24. The third electrolyte solution reservoir 38 may comprise the same electrolyte paint as that used in the electrolyte solution reservoir 24, applied in this case to the third ion exchange membrane 36.

Furthermore, the third ion exchange membrane 36 may comprise the same ion exchange resin as that present in the first ion exchange paint (of which the first ion exchange membrane 30 is formed), and may function as an ion exchange membrane similar to the first ion exchange membrane 30.

The fourth ion exchange membrane 40 may comprise an ion exchange paint applied to the third electrolyte solution reservoir 38 and containing the ion exchange resin comprising the second ion exchange membrane 26. The fourth ion exchange membrane 40 functions as an ion exchange membrane similar to the second ion exchange membrane 26.

An active electrode terminal 42 may be arranged on the other surface of the base sheet 18, and conduction may be established between the active electrode terminal 42 and the active electrode 22 of the active electrode assembly 12 via a through hole formed on the base sheet 18.

Similarly, a counter electrode terminal 44 may be disposed on the other surface of the base sheet 19, and conduction may be established between the counter electrode terminal 44 and the counter electrode 32 of the counter electrode assembly 14 via a through hole formed on the base sheet 19.

The DC electric power source 16 may be placed between the active electrode terminal 42 and the counter electrode terminal 44. The DC electric power source 16 may be a cell type battery comprising a first active electrode layer 46, a separator layer 47, and a second active electrode layer 48 laminated sequentially on one surface of the sheet like pad 18 by printing or the like. The first active electrode layer 46 of the DC electric power source 16 and the active electrode terminal 42 are directly connected to each other, and the second active electrode layer 48 and the counter electrode terminal 44 are connected to each other through a conductive paint coating film (a non-working conductive layer) 45 formed through an insulating paste layer 49.

Reference numeral 13 in FIG. 3 denotes a coupling belt for coupling the active electrode assembly 12 and the counter electrode assembly 14. The coating film 45 may also be applied to the coupling belt 13 and continue to the counter electrode terminal 44.

Thin-film batteries disclosed in JP 11-067236 A, US 2004/0185667 A1, and U.S. Pat. No. 6,855,441, each incorporated herein by reference in its entirety, may be used for the DC electric power source 16. However, the structure of the DC electric power source 16 is not limited to the thin-film batteries thus disclosed.

Further, an active agent solution reservoir may be configured so as to directly contact with an active electrode.

In addition, except a part constituted by a temperature responsive gel matrix, a portion of each of the electrolyte solution reservoirs, the active agent solution reservoir, and the ion exchange membranes may comprise a coating film. No limitations are placed on the configuration, however, and a plurality of membranes may be adhered to each other.

Furthermore, an active agent injecting device that injects an active agent solution through micro-needles by means of a pump, for example, may also be used as the active agent injecting device 120.

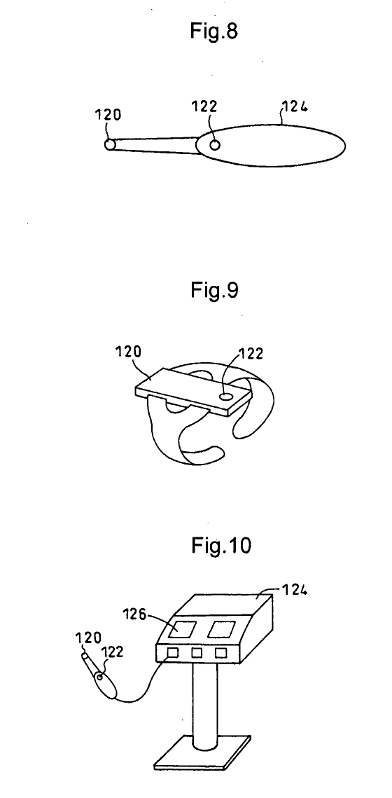

The active agent injecting device 120 may take a variety of geometric forms, including a rod shaped form shown in FIG. 8, a wrist band form shown in FIG. 9, and a stand type shown in FIG. 10.

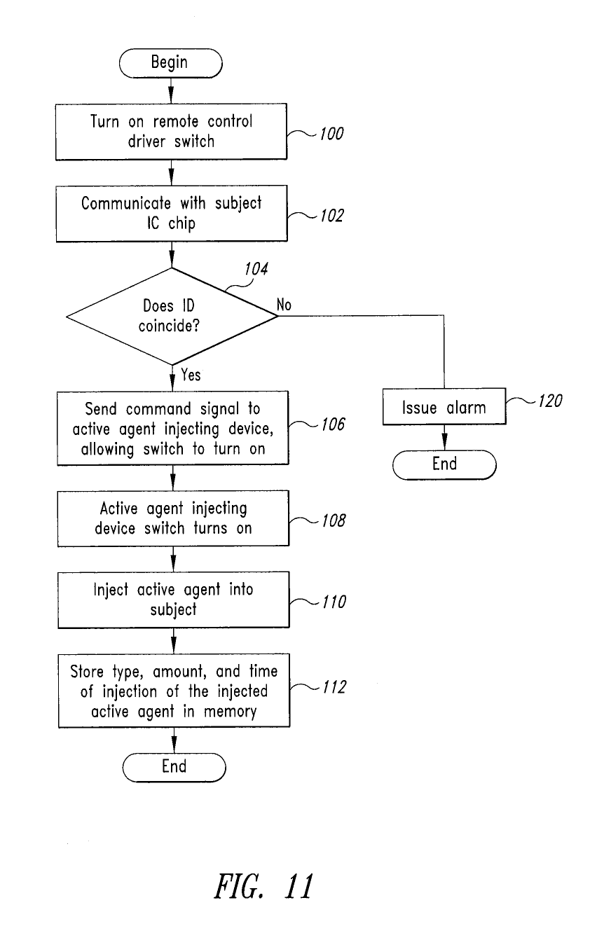

Operation will be described next with reference to FIG. 11.

When a driver switch 126 is turned on at 100, the remote controller 124 communicates with the IC chip 112 of the subject 100 at 102 to identify the ID of the subject. If the ID is determined to coincide with that of a subject to whom an administration instruction is given at 104, a command signal is sent to the active agent injecting device 120 at 106 to enable the switch 122 of the active agent injecting device 120 to be turned on so that an active agent can be injected into the subject 100. The command signal may be sent via a direct wired connection, or through a wireless connection. The active agent is injected into the subject 100 at 110 after the switch 122 is turned on at 108. The active agent type, amount of the active agent injected, and the time at which the active agent was injected are stored in a memory 128 rewritable with a built-in reader/writer at 112. Information stored in the memory 128 is thereafter collectively input to the server 130.

The memory 128 may store, for example, the name, amount, effective date, and manufacturer of an active agent loaded into the active agent injecting device 120, and the ID of a subject to whom the active agent is to be administered.

An alarm is issued at 120 if the ID does not coincide with that of the subject to whom an administration instruction is given at 104.

In each of the above embodiments, information about the administration of an active agent is stored in the memory 128 of the active agent injecting device 120, and is thereafter input to the server 130. Alternatively, each of the remote controller 124 and server 130 of the active agent injecting device 120 may be provided with a wireless device, or a portable phone may be used to send information about the administration of an active agent to the server 130 in real time.

In each of the above embodiments, although the subject's ID is stored in the IC chip 112, other methods may also be employed. For example, the following procedure may be adopted: a tape on which a one-dimensional or two-dimensional machine-readable symbol (e.g., barcode) is printed may be adhered to the subject, and the active agent injecting device 120 or the remoter controller 124 may be provided with a one dimensional or two dimensional symbol reader to read the machine-readable symbol.

The above description of illustrated embodiments, including what is described in the Abstract, is not intended to be exhaustive or to limit the embodiments to the precise forms disclosed. Although specific embodiments and examples are described herein for illustrative purposes, various equivalent modifications can be made without departing from the spirit and scope of the disclosure, as will be recognized by those skilled in the relevant art. The teachings provided herein of the various embodiments can be applied to other problem-solving systems devices, and methods, not necessarily the exemplary problem-solving systems devices, and methods generally described above.

For instance, the foregoing detailed description has set forth various embodiments of the systems, devices, and/or methods via the use of block diagrams, schematics, and examples. Insofar as such block diagrams, schematics, and examples contain one or more functions and/or operations, it will be understood by those skilled in the art that each function and/or operation within such block diagrams, flowcharts, or examples can be implemented, individually and/or collectively, by a wide range of hardware, software, firmware, or virtually any combination thereof. In one embodiment, the present subject matter may be implemented via Application Specific Integrated Circuits (ASICs). However, those skilled in the art will recognize that the embodiments disclosed herein, in whole or in part, can be equivalently implemented in standard integrated circuits, as one or more computer programs running on one or more computers (e.g., as one or more programs running on one or more computer systems), as one or more programs running on one or more controllers (e.g., microcontrollers) as one or more programs running on one or more processors (e.g., microprocessors), as firmware, or as virtually any combination thereof, and that designing the circuitry and/or writing the code for the software and or firmware would be well within the skill of one of ordinary skill in the art in light of this disclosure.

In addition, those skilled in the art will appreciate that the mechanisms taught herein are capable of being distributed as a program product in a variety of forms, and that an illustrative embodiment applies equally regardless of the particular type of signal bearing media used to actually carry out the distribution. Examples of signal bearing media include, but are not limited to, the following: recordable type media such as floppy disks, hard disk drives, CD ROMs, digital tape, and computer memory; and transmission type media such as digital and analog communication links using TDM or IP based communication links (e.g., packet links).

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet including but not limited to U.S. Provisional Patent Application Ser. No. 60/719,632, filed Sep. 21, 2005, and Japan Patent Application No. 2005-237755, filed Aug. 18, 2005, are incorporated herein by reference, in their entirety.

Aspects of the embodiments can be modified, if necessary, to employ systems, circuits, and concepts of the various patents, applications, and publications to provide yet further embodiments.

These and other changes can be made to the embodiments in light of the above-detailed description. In general, in the following claims, the terms used should not be construed to limit the invention to the specific embodiments disclosed in the specification and the claims, but should be construed to include all possible embodiments along with the full scope of equivalents to which such claims are entitled. Accordingly, the scope of the invention shall only be construed and defined by the scope of the appended claims.

What is claimed is:

1. A method of managing active agent usage, the method comprising managing an active agent to be administered based on actual drive information from an active agent injecting device.

2. The method of managing active agent usage according to claim 1 wherein the active agent injecting device validates a drive signal after an identity (ID) of a subject, to whom the active agent is to be administered, is authenticated.

3. The method of managing active agent usage according to claim 2, further comprising:
physically coupling an ID device having ID Information to the subject before the ID of the subject is authenticated.

4. The method of managing active agent usage according to claim 3 wherein physically coupling the ID device to the subject includes implanting the ID device in the subject.

5. The method of managing active agent usage according to claim 2 wherein the active agent injecting device comprises an iontophoresis device.

6. A device for managing active agent usage, comprising:
an active agent injecting device driven by a drive signal; and
a server that manages active agent usage based on actual drive information from the active agent injecting device.

7. The device for managing active agent usage according to claim 6 wherein the server manages an active agent type, an amount, and an administration timing for an active agent administered to a subject, on a per subject basis.

8. The device for managing active agent usage according to claim 7 wherein information on an active agent administered by the active agent injecting device is stored in the active agent injecting device and is later input into the server.

9. The device for managing active agent usage according to claim 7 wherein information on an active agent administered by the active agent injecting device is sent to the server by radio communication.

10. The device for managing active agent usage according to claim 6 wherein information on an active agent administered by the active agent injecting device is stored in the active agent injecting device and is later input into the server.

11. The device for managing active agent usage according to claim 6 wherein information on an active agent administered by the active agent injecting device is sent to the server by radio communication.

12. An active agent injecting device wherein a drive signal thereof is validated after an identity (ID) of a subject, to whom an active agent is to be administered, is authenticated.

13. A method of managing delivery of an active agent from an active agent delivery device, the method comprising:
reading identifying information from an identification device physically coupled to a subject;
determining at least one parameter of an active agent delivery protocol based at least in part on the read identifying information; and
applying a drive signal to at least a portion of the active agent delivery device to deliver the active agent to the subject according to the active agent delivery protocol.

14. The method of claim 13 wherein reading identifying information from an identification device includes automatically reading the identifying information from the identification device.

15. The method of claim 14 wherein automatically reading the identifying information includes optically reading a machine-readable symbol carried by the identification device.

16. The method of claim 13 wherein determining at least one parameter of an active agent delivery protocol includes determining at least one of an active agent type, an amount or an administrative schedule.

17. The method of claim 13 wherein applying a drive signal includes providing drive information from an active agent management server to the active agent delivery device, the active agent management server serving a plurality of active agent delivery devices.

18. The method of claim 13 wherein applying a drive signal includes controlling a voltage and current supplied to at least one electrode of the active agent delivery device.

19. The method of claim 13, further comprising:
selecting the active agent delivery protocol based on the read identifying information.
